(19) United States
(12) Patent Application Publication
(10) Pub. No.: US 2011/0208257 A1
(43) Pub. Date: Aug. 25, 2011

(54) CRANIAL ELECTROSTIMULATION METHOD, EQUIPMENT AND ELECTRODE UNIT

(75) Inventor: Jaen Frederik LABUSCHAGNE, Western Cape Province (ZA)

(73) Assignee: TO BE FIRST AG, Zug (CH)

(21) Appl. No.: 13/119,167

(22) PCT Filed: Sep. 17, 2009
(86) PCT No.: PCT/IB2009/006863

§ 371 Date: Mar. 15, 2011

(30) Foreign Application Priority Data
Sep. 18, 2008 (GB) 0817092.0

(51) Int. Cl. A61N 1/36

(52) U.S. Cl. 607/3; 607/45

(57) ABSTRACT

A method of conducting cranial electrostimulation on a patient is provided as well as equipment therefore. Suitable electrodes are attached to the head of the patient and electrostimulation currents are created in response to pulses applied to the electrodes. Multiple electrodes are employed and the electrostimulation currents are caused to flow between different pairs of electrodes of the multiplicity thereof sequentially in either a random order or a predetermined order. Typically, the multiple electrodes are arranged in two sets thereof in which instance the different pairs of electrodes are selected one from one set and one from the other set so that current flows between the electrodes of only one pair at any one time. The sets of electrodes are generally engaged with the ears of a patient. The polarity of the pulses applied to the various pairs of electrodes may be varied sequentially or randomly.

### FIELD OF THE INVENTION

This invention relates to a cranial electrostimulation method such as that often referred to as electrotherapy and to equipment for use in carrying out the method, especially to an electrode unit forming part of the equipment in use. More especially, but not necessarily exclusively, the invention relates to an electrostimulation method in the form of what is known as cranial electro-biologic stimulation that is currently, as far as applicant is aware, typically applied between electrodes clipped to the earlobes of a patient receiving treatment.

It is to be understood that the term “patient” as used in this specification is intended to be interpreted broadly as being any person being subjected to electrostimulation irrespective of whether the underlying reason for applying such electrostimulation has curative intentions or not. In this regard it is noted that electrostimulation is often used with the intention of simply improving a patient's quality of life such as by improving the patient's mood, emotions, attitude or cognitive capabilities.

### BACKGROUND TO THE INVENTION

Cranial electro-biologic stimulation (herein referred to as CES) applies gentle micro-current pulses to the brain (in the hypothalamic area) using a pair of ear-clip electrodes that attach to the earlobes. It is widely accepted that CES stimulates the brain to manufacture neurotransmitters, like endorphins, which improve moods, emotions and cognitive capabilities. Cranial electrostimulation has also been proposed for treatment following a stroke, brain trauma, certain heart disorders, high blood pressure, jet leg, motion sickness and dementia.

The signals apparently normalise the electrical output of the brain. CES has thus been used or tested to treat substance dependence, depression and anxiety. It has been noted in at least some instances that CES has equal or greater efficacy for the treatment of depression when compared to antidepressant medications, with fewer side effects.

The mechanism by which CES produces its effects is not yet fully understood. It is postulated that the stimulation of brain tissue causes increased amounts of neurotransmitters to be released, specifically serotonin, beta endorphin, and noradrenaline. It is believed that these neurotransmitters in turn permit a return to normal biochemical homeostasis of the limbic system of the brain that may have been imbalanced by a stress-related condition.

Treatments that have typically been used range from 10 to 30 minutes in duration although they may extend up to 1½ hours depending on the electrical current configuration. Typically the currents employed would be applied in pulse form with a pulse width in the range of from about 1 to about 500 milliseconds (ms) at a frequency of from about 0.1 Hertz (Hz) to about 500 Hz with the current being typically less than 1 milliampere (mA) and more typically about 300 to 700 microamperes (μA), although currents of up to about 2.5 milliampere (mA) have also been used.

### OBJECT OF THE INVENTION

It is an object of this invention to provide a cranial electrostimulation method and equipment including an electrode unit aimed at having an enhanced overall effect on a patient.

### SUMMARY OF THE INVENTION

In accordance with one aspect of this invention there is provided a method of conducting cranial electrostimulation on a patient comprising attaching suitable electrodes to the head of the patient and causing electrostimulation currents to be created in response to pulses applied to the electrodes, the method being characterised in that multiple electrodes are attached to the head of a patient and in that said electrostimulation currents are caused to flow between different pairs of electrodes of said multiplicity thereof sequentially in either a random order or a predetermined order.

Further features of this aspect of the invention provide for the multiple electrodes to be arranged in two sets thereof in which instance the pairs of electrodes are selected one from one set and one from the other set so that current flows between the electrodes of only one pair at any one time; for the sets of electrodes to be shaped to engage the ears of a patient, in particular, behind the pinna and earlobe of the ear or, alternatively, around the entrance to the auditory canal, or within the auditory canal; for each set of electrodes to comprise three, four, five or more individual electrodes spaced apart on a carrier; for the method to include playing music by way of earphones such that the music is audible to a patient during treatment; and for the polarity of pulses applied to the various pairs of electrodes to be varied sequentially or randomly.

Still further features of this aspect of the invention provide for the pulse frequency and width to be dependent on the rhythm, beat or tempo of music that is audible to the patient such that the average current remains generally constant over the range of different frequencies; and for the electrical pulses to fall within the general ambit considered as cranial electro-biologic stimulation.

In accordance with a second aspect of the invention there is provided equipment for the implementation of a method as defined above, said equipment comprising a cranial electrostimulation pulse generator and associated electrodes for applying pulses generated by the pulse generator to the head of a patient, the equipment being characterised in that it includes multiple electrodes and a controller for connecting the pulse generator to different pairs of electrodes of said multiple electrodes sequentially in either a random order or a predetermined order to cause, in use, electrostimulation currents to flow sequentially between the different pairs of electrodes.

Further features of the second aspect of the invention provide for the multiple electrodes to be arranged in two sets thereof as set out in the further features to the first aspect of the invention; and for the controller to be connected to a predetermined number of predetermined pairs of electrodes to selectively and sequentially energize any one pair of electrodes at any one time or, alternatively, for the controller to be connected to all of the electrodes and to have selector means for selecting one electrode of each set thereof at any one time.

Other features of the equipment correspond to the further features of the first aspect of the invention defined above and, in particular, include the provision of a sound signal generator and at least one associated loudspeaker for converting output from the sound signal generator into audible sound. The at least one loudspeaker is preferably a pair of earphones and the cranial electrostimulation pulse generator and sound signal generator may be built into a single unit, but are not necessarily thus combined.

Any such single unit may be a portable unit that is preferably battery powered

The unit preferably includes a data base containing a selection of different musical numbers and treatment programmes that can be selected by a user by way of manually operable controls and an interface display screen for facilitating a selection being made.

In accordance with a third aspect of the invention there is provided an electrode unit comprising an electrode carrier and electrode for conducting cranial electrostimulation on a patient, the electrode unit being characterised in that multiple individually operable electrodes are provided on the carrier.

Further features of this third aspect of the invention provide for the carrier to be configured to be held in contact with a patient's ear with the multiple electrodes in contact with the skin of the ear; for the carrier to be attached to an earphone unit of the type shaped to be held against the ear a short distance outwards from the entrance to the auditory canal or in the entrance to the auditory canal or within the auditory canal; for the electrode carrier to be of arcuate shape that can be swung outwards away from the earphone such that it can be swung inwards to engage the rear face of the pinna of a person's ear to hold the electrode unit in position; and for the individual electrodes to be covered by an electrically conductive patch or the like.

It is to be noted that part or all of the electrostimulation pulse generator and any sound signal generator may be embodied in an electrode unit or earphone or with some components located in each of the two electrode units or earphones of a pair thereof.

In order that the above and other features of the invention may be more fully understood different embodiments thereof will be described below with reference to the accompanying drawings.

### BRIEF DESCRIPTION OF THE DRAWINGS

In the drawings:—

FIG. 1 is a block diagram of one embodiment of method and equipment for use in exercising this invention;

FIG. 2 is a perspective illustration of a portable version of equipment operating on the basis of the block diagram of FIG. 1;

FIG. 3 is an elevation from the outside of a multi-electrode earphone having electrodes arranged to contact the ear externally on or adjacent the pinna and earlobe;

FIG. 4 illustrates in FIG. 4a the inside of a multielectrode earphone illustrated in FIG. 3; in FIG. 4b the multielectrode earphone open; and in FIG. 4c felt covers for the electrodes;

FIG. 5 illustrates in FIG. 5a the multielectrode earphone open and about to be installed on an ear; in FIG. 5b the earphone installed on the ear, and in FIG. 5c zones in which the various electrodes contact the ear;

FIG. 6 illustrates an alternative form of multi-electrode earphone for introduction into the entrance of the auditory canal;

FIG. 7 is a graphic illustration of a sequence of different coupled pairs of electrodes in a multielectrode arrangement;

FIG. 8 is a block diagram of one arrangement for selecting channels and polarity in a multielectrode arrangement; and,

FIG. 9 is a block diagram of an alternative arrangement for selecting channels and polarity in a multielectrode arrangement.

### DETAILED DESCRIPTION WITH REFERENCE TO THE DRAWINGS

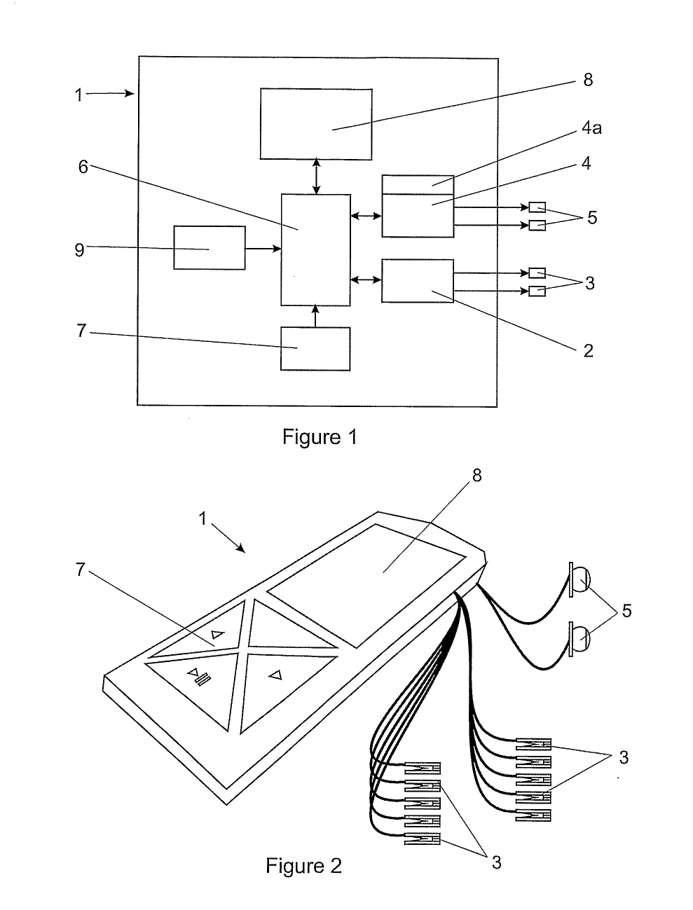

In the embodiment of the invention illustrated in FIGS. 1 and 2, the method of the invention is implemented by way of equipment that, in one instance, is a portable battery powered unit (1) that may take the shape and configuration of a pocket sized therapy unit, as illustrated in FIG. 2.

The therapy unit is configured to carry out CES on a patient by the application of appropriate electrical pulses to the ears simultaneously with the playing of music that is audible to the patient. The equipment thus comprises a cranial electrostimulation pulse generator (2) and associated two sets of multiple electrodes (3) for applying pulses generated by the pulse generator to the ears of a patient, and a sound signal generator (4) and a pair of earphones (5) for converting output from the sound signal generator into audible sound. The sound signal generator may have associated with it a data base containing a selection of different musical numbers that can be selected by a user or by selecting a predetermined treatment programme or session. The data base is indicated by numeral (4a) in FIG. 1.

The outputs from the electrostimulation pulse generator and sound signal generator are controlled by a controller in the form of a microprocessor (6) that may be user operated by a user button interface (7). The unit has a display screen (8) for assisting in programme selection in very much a conventional manner. Obviously the display screen could be replaced by any other suitable indicator means such as an array of light emitting diodes or the like. A battery charger circuit (9) is also provided.

The above arrangement is such that the electrostimulation pulse generator produces electrical pulses having a frequency and pulse width selected to provide a required microcurrent across the head, in use, such microcurrent typically being of the order of 500 to 750 μA and, in any event, less than 1 mA. The frequency could range from 0.5 Hz to about 3 Hz and the pulse width could range from about 125 ms at a frequency of 0.5 Hz to about 75 ms at a frequency of 3 Hz. The microcurrent across the head is, in any event, generally maintained at a substantially constant average current.

The pulses (or possibly sequences of pulses in the event that predetermined sequences of optionally different pulses are employed) are, in this embodiment of the invention, generated at a frequency that is dependant on the tempo of music that is being played at any one time and that is audible to the patient by way of the earphones. Of course, in order to achieve a substantially constant average current, the pulse width of the pulses will also vary with variation in frequency. The microprocessor monitors the tempo of the music and controls the generation of the electrical pulses on the basis thereof.

Whilst not in any way being limiting to the scope of the invention it is proposed that the following could operate effectively:—

At an audio tempo of 30 beats per minute a frequency of 0.5 Hz could be employed with a pulse width of 125 ms;

at an audio tempo of 60 beats per minute a frequency of 1.0 Hz could be employed with a pulse width of 115 ms;

at an audio tempo of 90 beats per minute a frequency of 1.5 Hz could be employed with a pulse width of 105 ms;

at an audio tempo of 120 beats per minute a frequency of 2.0 Hz could be employed with a pulse width of 95 ms;

at an audio tempo of 150 beats per minute a frequency of 2.5 Hz could be employed with a pulse width of 85 ms; and,

at an audio tempo of 180 beats per minute a frequency of 3.0 Hz could be employed with a pulse width of 75 ms.

The microprocessor is also programmed to provide treatment programmes or sessions of a predetermined duration, as is well known in the art, and the duration would generally be somewhere between 10 minutes and 30 minutes but may extend up to an hour or even an hour and a half. Information as to any particular treatment session to be chosen, musical numbers and the like can be displayed on the display screen (8) provided on the therapy unit.

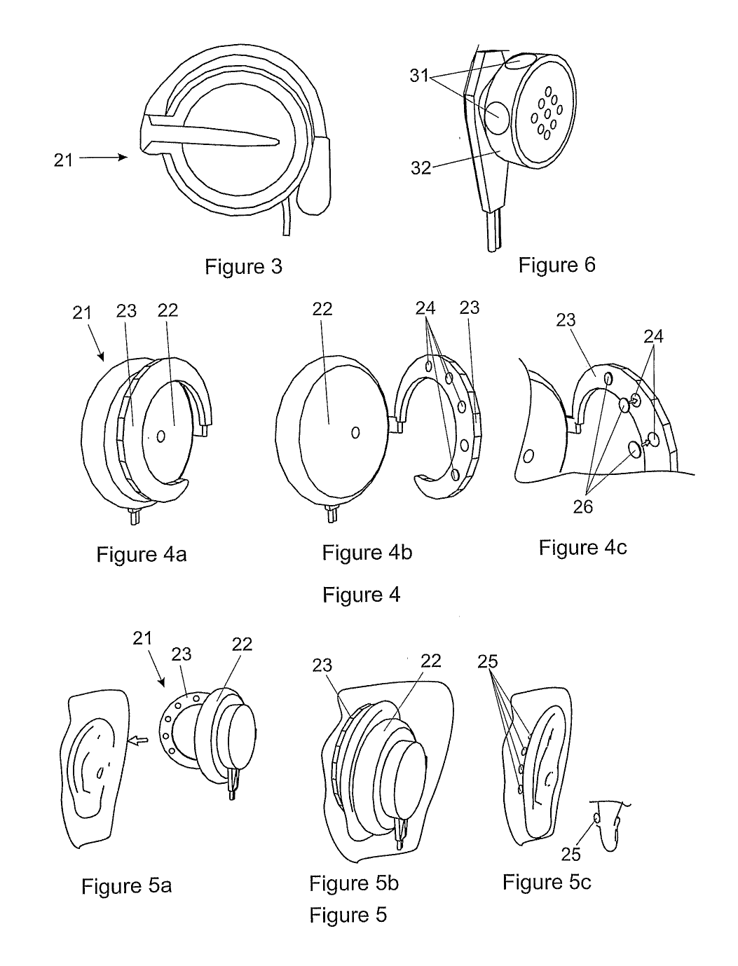

Reverting now to the nature of the sets of multielectrodes, each set is made, in this embodiment of the invention, in the form of a multielectrode unit that is illustrated in FIGS. 3 to 5 of the accompanying drawings. In this instance an earphone unit (21) has an earphone (22) of the type shaped to be held against the ear a short distance outwards from the entrance to the auditory canal and an arcuate electrode carrier (23) that can be swung outwards away from the sound emitting face of the earphone.

The electrode carrier can thus be swung inwards to engage the rear face of the pinna of a person's ear to hold the earphone unit in position. In the operative position the multiple electrodes (3) in the face of the arcuate electrode carrier contact the rear of the pinna of the ear at arcuately spaced positions that are indicated by numeral (25) in FIG. 5c. Each individual electrode is preferably covered by an electrically conductive felt patch (26) or the like, as shown in FIG. 4c.

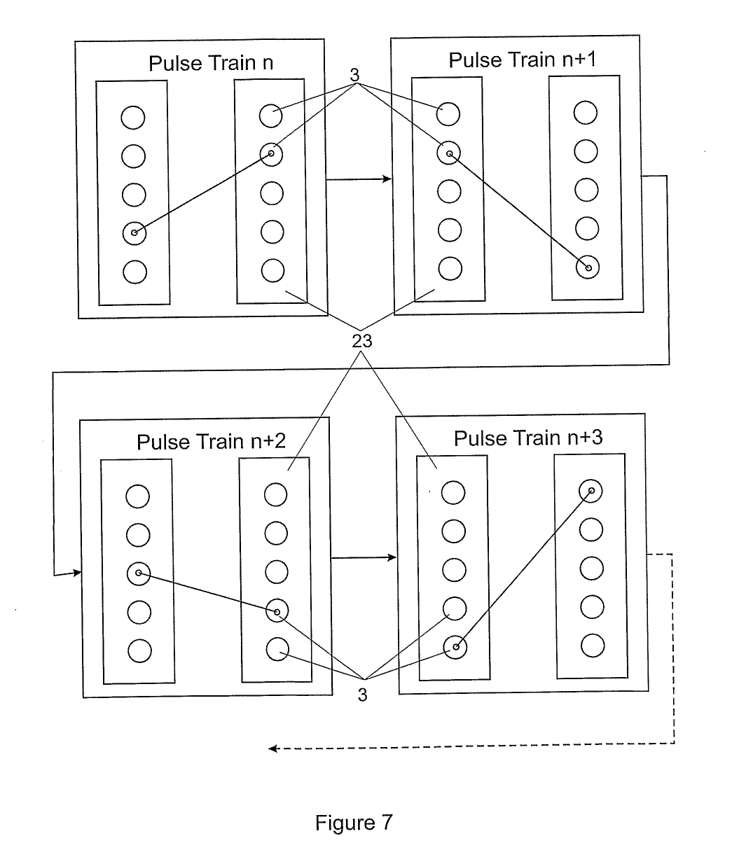

In this variation of the invention, the arrangement is such that current is only induced between one electrode of each earphone unit at any one time and different pairs of cooperating electrodes are selected sequentially or randomly via the microprocessor. FIG. 7 indicates some sequential connections between single electrodes of each multiplicity thereof simply by way of example.

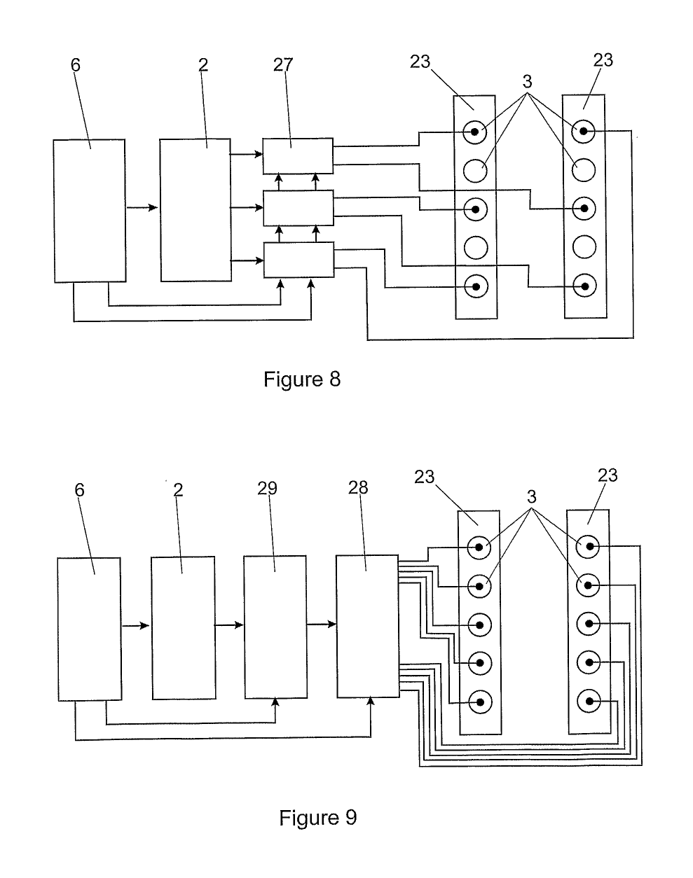

In one type of arrangement that is illustrated in FIG. 8, each electrode pair to be used in a circuit is allocated a channel (27) and the microprocessor can activate a channel and select a direction of the current by setting the polarity of the electrodes of a pair in a random or preprogrammed order. This arrangement may be rather hardware intensive in the event that many channels are to be used.

Another type of arrangement is illustrated in FIG. 9 in which a multiplexing type of arrangement is illustrated and the microprocessor selects pairs of electrodes of each of the sets randomly and each circuit is closed utilising an analogue switch unit (28). In this arrangement the polarity is controlled by the microprocessor by way of a direction control unit (29).

It is envisaged that the use of multielectrode units will most likely provide different electrical paths through the cranial region of a patient's head with a likely enhancement of the therapeutic value being obtained consequent on a treatment.

FIG. 6 illustrates one alternative multielectrode earphone unit in which the multiple electrodes (31) are spaced apart on a cylindrical insulating surface (32) of the type of earphone adapted to be received in the entrance to the auditory canal. Of course, the same type arrangement can be embodied in the smaller type of earphones that are configured to be received within the auditory canal and that are sometimes referred to as canal phones or earbuds.

In each instance in which multielectrode earphone units are employed, the pulses of electrical energy may be varied according to the tempo of the music being played although the use of music in this way, or the use of music at all, is not required in exercising the present invention.

It will be understood that numerous variations may be made to the various aspects of the invention described above without departing from the scope hereof. In particular, numerous different arrangements of electrode are possible within the scope of the invention, those identified above simply being examples.

Also, as regards the equipment generally, the electrostimulation pulse generator and sound signal generator do not need to be embodied in a single unit, especially when used in a treatment centre or studio and, indeed, the sound signal generator need not be present at all, depending on the objectives to be achieved and the duty that the equipment is required to perform.

1. A method of conducting cranial electrostimulation on a patient comprising attaching suitable electrodes to the head of the patient and causing electrostimulation currents to be created in response to pulses applied to the electrodes, the method being characterised in that multiple electrodes are attached to the head of a patient and in that said electrostimulation currents are caused to flow between different pairs of electrodes of said multiplicity thereof sequentially in either a random order or a predetermined order.

2. A method as claimed in claim 1 in which the multiple electrodes are arranged in two sets thereof in which instance the said different pairs of electrodes are selected one from one set and one from the other set so that current flows between the electrodes of only one pair at any one time.

3. A method as claimed in claim 2 in which the sets of electrodes are engaged with the ears of a patient in areas selected from behind the pinna and earlobe of the ear, around the entrance to the auditory canal of the ear and within the auditory canal of the ear.

4. A method as claimed in claim 2 in which each set of electrodes comprises three, four, five or more individual electrodes spaced apart on a carrier (23).

5. A method as claimed in claim 1 in which the method includes playing music by way of earphones (5) such that the music is audible to a patient during treatment.

6. A method as claimed in claim 1 in which the polarity of pulses applied to the various pairs of electrodes is varied sequentially or randomly.

7. A method as claimed in claim 5 in which the pulse frequency and width are dependent on the rhythm, beat or tempo of music that is audible to the patient such that the average current remains generally constant over the range of different frequencies.

8. A method as claimed in claim 1 in which the electrical pulses fall within the general ambit considered as cranial electro-biologic stimulation.

9. Equipment for the implementation of a method as claimed in claim 1, said equipment comprising a cranial electrostimulation pulse generator and associated electrodes for applying pulses generated by the pulse generator to the head of a patient, the equipment being characterised in that it includes multiple electrodes and a controller for connecting the pulse generator to different pairs of electrodes of said multiple electrodes sequentially in either random order or a predetermined order to cause, in use, electrostimulation currents to flow between the different pairs of electrodes.

10. Equipment as claimed in claim 9 in which the multiple electrodes are arranged in two sets thereof.

11. Equipment as claimed in claim 9 in which the controller is connected to a predetermined number of predetermined pairs of electrodes to selectively and sequentially energize any one pair of electrodes at any one time.

12. Equipment as claimed in claim 9 in which the controller is connected to all of the electrodes and has selector means for selecting electrodes to be interconnected and at any one time.

13. An electrode unit for use in equipment as claimed in claim 9 comprising an electrode carrier and electrode for conducting cranial electrostimulation on a patient, the electrode unit being characterised in that multiple individually operable electrodes are provided on the carrier.

14. An electrode unit as claimed in claim 13 in which the carrier is configured to be held in contact with a patient's ear with the multiple electrodes in contact with the skin of the ear.

15. An electrode unit as claimed in claim 13 in which for the carrier is attached to an earphone unit of the type shaped to be held against the ear a short distance outwards from the entrance to the auditory canal or in the entrance to the auditory canal or within the auditory canal.
